(19) United States
(12) Patent Application Publication
(10) Pub. No.: US 2009/0270798 A1
(43) Pub. Date: Oct. 29, 2009

(54) METHOD AND INSULIN PUMP FOR PROVIDING A BOLUS ALERT

(75) Inventor: Steven P. Jones, Rochester, MN (US)

Correspondence Address: CANTOR COLBURN LLP - IBM ROCHESTER DIVISION, 20 Church Street, 22nd Floor, Hartford, CT, 06103, US

(73) Assignee: INTERNATIONAL BUSINESS MACHINES CORPORATION, Armonk, NY (US)

(21) Appl. No.: 12/111,245

(22) Filed: Apr. 29, 2008

(51) Int. Cl. A61M 31/00

(52) U.S. Cl. 604/67

(57) ABSTRACT

A method of providing a bolus alert for an insulin pump user includes determining a bolus interval for an insulin pump user, establishing a bolus amount for the bolus interval, determining a bolus amount taken by the insulin pump user during the bolus interval, and providing an alert if the bolus amount taken by the insulin pump user differs from the bolus amount established for the bolus interval.

### BACKGROUND OF THE INVENTION

1. Field of the Invention

This invention relates to the art of insulin pumps and, more particularly, to a method of providing a bolus alert to an insulin pump user.

2. Description of Background

Various insulin pumps are currently available for use by diabetics. An insulin pump is a device that periodically dispenses very small amounts of insulin according to a preprogrammed profile set by a user to cover basal insulin needs. In addition to providing for basal insulin needs, the insulin pump can also provide a bolus dose as necessary.

When a person with diabetes consumes food, a bolus dose of insulin is required to counteract a blood sugar increase associated with the consumption of carbohydrates. More specifically, following a meal or snack, a person with diabetes must estimate an amount of carbohydrates consumed. After estimating the amount of carbohydrates, the insulin pump is programmed to delver a bolus dose of insulin to cover the consumed carbohydrates. Because the amount of the bolus can differ from meal to meal and from individual to individual, the insulin pump is not preprogrammed to administer a bolus dosage.

It is quite easy for a person with diabetes to forget to program a bolus at mealtime. The failure to program the bolus can result from a lapse of memory or simply through error, such as failure to properly push any required buttons on the insulin pump. If the bolus is not administered, blood sugar levels can rise to unhealthy or potentially dangerous levels. Towards that end, many insulin pumps provide an alert at mealtimes reminding the person with diabetes that a bolus may be due. However, while many pumps can provide a reminder of a forgotten bolus, existing pumps do not provide an alert regarding whether the amount of the bolus is within normal parameters for the user of the insulin pump.

### SUMMARY OF THE INVENTION

The shortcomings of the prior art are overcome and additional advantages are provided through the provision of a method of providing a bolus alert for an insulin pump user. The method includes determining a bolus interval for an insulin pump user, establishing a bolus amount for the bolus interval, determining a bolus amount taken by the insulin pump user during the bolus interval, and providing an alert if the bolus amount taken by the insulin pump user differs from the bolus amount established for the bolus interval.

Additional features and advantages are realized through the techniques of exemplary embodiments of the present invention. Other embodiments and aspects of the invention are described in detail herein and are considered a part of the claimed invention. For a better understanding of the invention with advantages and features, refer to the description and to the drawings.

### TECHNICAL EFFECTS

As a result of the summarized invention, technically we have achieved a solution which alerts insulin pump users to potential over or under dosage of insulin taken for a particular bolus interval.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates an insulin pump including a bolus alert in accordance with an exemplary embodiment of the invention;

FIG. 2 is a flow diagram illustrating a method of signaling a bolus alert in accordance with an exemplary embodiment of the invention.

The detailed description explains the exemplary embodiments of the invention, together with advantages and features, by way of example with reference to the drawings.

### DETAILED DESCRIPTION OF THE INVENTION

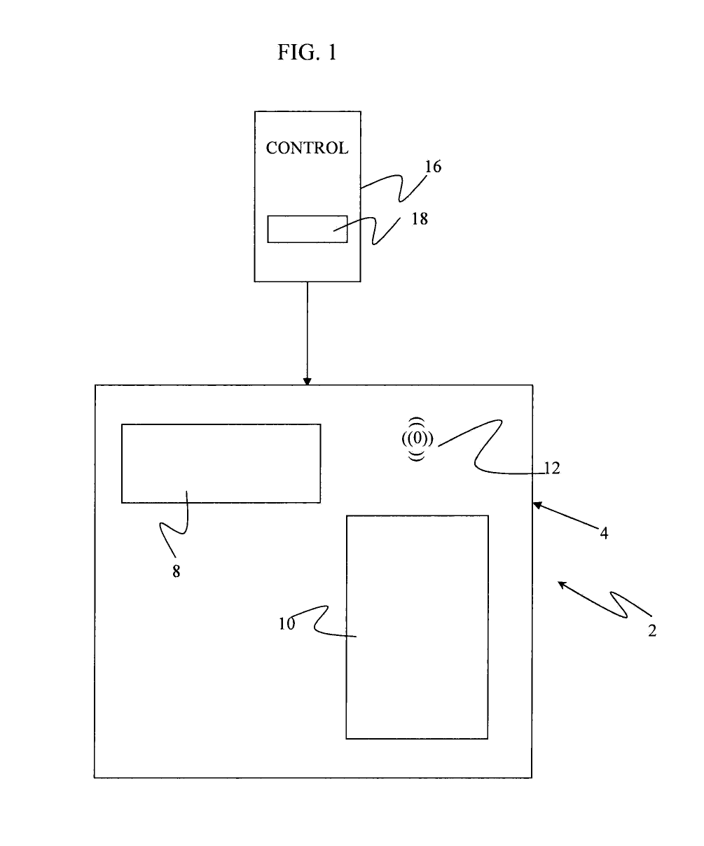

With initial reference to FIG. 1, an insulin pump constructed in accordance with an exemplary embodiment of the present invention is indicated generally at 2. As shown, insulin pump 2 includes a main body 4 that houses a display 8, a keypad 10 and an alarm 12. As will be discussed more fully below, in addition to programming various options for insulin pump 2, display 8 provides a visual alert and/or alarm 12 provides an audible alert when an administered bolus is less than an expected bolus amount. Towards that end, insulin pump 2 includes a processor or CPU 16 having a programmable memory 18. Processor 16 receives inputs through keypad 10 and/or from external software via a wireless or wired connection regarding bolus parameters for the user. The bolus parameters enable processor 16 to determine if an administered bolus is less than an expected bolus for a particular bolus interval.

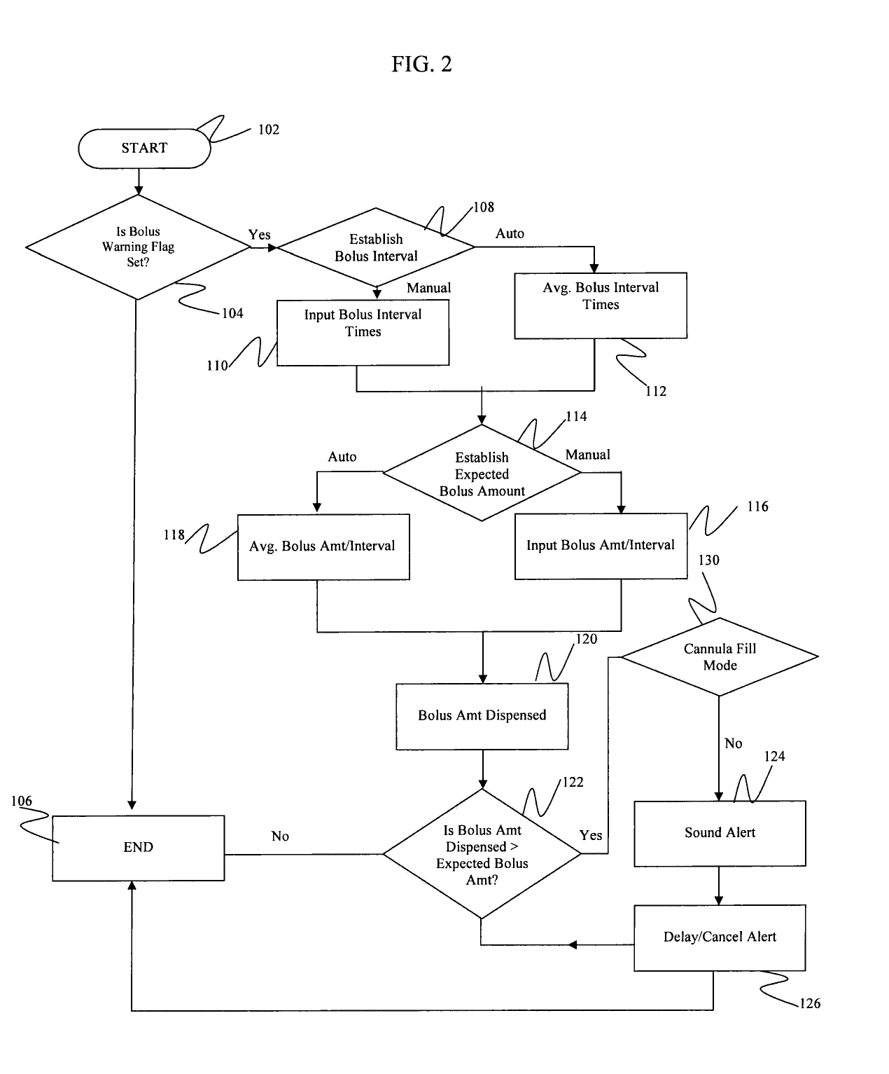

Reference will now be made to FIG. 2 in describing a method 100 of generating a bolus warning starting at block 102. Initially, a determination is made whether a bolus warning flag is set in block 104. If the flag is not set, method 100 ends at block 106. If however the bolus warning flag is set in block 102, a bolus interval(s) is established in block 108. The bolus interval is a defined time period that typically corresponds to meal times for the user of insulin pump 2. The bolus interval(s) can be input manually as indicated in block 110 through, for example, keypad 10, or determined automatically as indicated in block 112. That is, over time, processor 16 learns bolus intervals for the user of insulin pump 2 by averaging bolus intervals over time.

After establishing the bolus interval(s) in block 108, an expected bolus amount for each interval is determined as indicated in block 114. The expected bolus amount can be determined manually as indicated in block 116, for example, input through keypad 10, or automatically as indicated in block 118. Processor 16 stores bolus amounts for each bolus interval in memory 18. The bolus amounts stored in memory 18 are averaged to determine an expected bolus amount for each bolus interval.

Once a bolus is dispensed, a determination is made in block 120 whether the bolus is less than the expected bolus amount determined in block 114. If the dispensed bolus is equal to or greater than the expected bolus, the alert process ends at block 104. If however, the dispensed bolus is less than the expected bolus, an alarm or alert is provided at block 124. At this point, the user of insulin pump 2 can delay the alert in block 126, if for example the disparity between the dispensed bolus and expected bolus is with good reason, or end the alert and take the bolus.

There are a number of reasons for a disparity between a dispensed bolus and an expected bolus. For example, if the user is interrupted while setting the bolus, the amount taken could be less than anticipated. Also, if the insulin reservoir is depleted before a full bolus is dispensed, a disparity may exist. Other reasons for a disparity include replacing and filling a cannula with insulin during a bolus period. In such a case, the user enters a cannula fill mode as shown in block 130 to override any alert. A disparity may also occur is a correction bolus is administered during a bolus interval. For example, if the user takes a correcting bolus during a bolus period, the correcting bolus is very likely less than the expected bolus. A correcting bolus is taken when a blood glucose reading at a particular time is higher than the blood glucose reading should be at the particular time. A correcting bolus may be taken at any time, including during a bolus interval. The insulin pump user receiving an alarm when taking a correcting bolus during a bolus interval would simply reset the alarm.

At this point it should be appreciated that exemplary embodiments of the present invention provide an insulin pump with a bolus warming to alert users that a bolus may be less than an expected bolus for a particular bolus interval. The alert provides a reminder to take the bolus if forgotten, that is the dispensed bolus is zero units or delay the alert if the less than expected bolus occurs for a reason such as, interruption, cannula fill or a correcting bolus is taken.

The capabilities of the present invention can be implemented in software, firmware, hardware or some combination thereof.

As one example, one or more aspects of the present invention can be included in an article of manufacture (e.g., one or more computer program products) having, for instance, computer usable media. The media has embodied therein, for instance, computer readable program code means for providing and facilitating the capabilities of the present invention. The article of manufacture can be included as a part of a computer system or sold separately.

Additionally, at least one program storage device readable by a machine, tangibly embodying at least one program of instructions executable by the machine to perform the capabilities of the present invention can be provided.

The flow diagrams depicted herein are just examples. There may be many variations to these diagrams or the steps (or operations) described therein without departing from the spirit of the invention. For instance, the steps may be performed in a differing order, or steps may be added, deleted or modified. All of these variations are considered a part of the claimed invention.

While the preferred embodiment to the invention has been described, it will be understood that those skilled in the art, both now and in the future, may make various improvements and enhancements which fall within the scope of the claims which follow. These claims should be construed to maintain the proper protection for the invention first described.

1. A method of providing a bolus alert for an insulin pump user, the method comprising:
determining a bolus interval for an insulin pump user;
establishing an expected bolus amount for the bolus interval;
determining a bolus amount taken by the insulin pump user during the bolus interval; and
providing an alert if the bolus amount taken by the insulin pump user differs from the expected bolus amount for the bolus interval.

2. The method of claim 1, wherein establishing the expected bolus amount for the bolus interval includes averaging bolus amounts for similar bolus intervals.

3. The method of claim 1, wherein establishing the expected bolus amount for the bolus interval includes inputting the expected bolus amount into a memory of the insulin pump.

4. The method of claim 1, further comprising: delaying the alert if the bolus amount taken by the insulin pump user is known to differ from the bolus amount established by the insulin pump.

5. The method of claim 1, further comprising: a cannula fill mode, the alert being bypassed in the cannula fill mode.
